SEQUENCE LISTING (15 sequences)

SEQ ID NO: 1 - protein, 4 aa, Artificial Sequence
  Cell adhesion peptide
RGDG

SEQ ID NO: 2 - protein, 4 aa, Artificial Sequence
  Cell adhesion peptide
RGDA

SEQ ID NO: 3 - protein, 4 aa, Artificial Sequence
  Cell adhesion peptide
RGDV

SEQ ID NO: 4 - protein, 4 aa, Artificial Sequence
  Cell adhesion peptide
RGDS

SEQ ID NO: 5 - protein, 4 aa, Artificial Sequence
  Cell adhesion peptide
RGDT

SEQ ID NO: 6 - protein, 4 aa, Artificial Sequence
  Cell adhesion peptide
RGDF

SEQ ID NO: 7 - protein, 4 aa, Artificial Sequence
  Cell adhesion peptide
RGDM

SEQ ID NO: 8 - protein, 4 aa, Artificial Sequence
  Cell adhesion peptide
RGDP

SEQ ID NO: 9 - protein, 4 aa, Artificial Sequence
  Cell adhesion peptide
RGDN

SEQ ID NO: 10 - protein, 5 aa, Artificial Sequence
  Cell adhesion peptide
YIGSR

SEQ ID NO: 11 - protein, 5 aa, Artificial Sequence
  Cell adhesion peptide
PDSGR

SEQ ID NO: 12 - protein, 4 aa, Artificial Sequence
  Cell adhesion peptide
REDV

SEQ ID NO: 13 - protein, 5 aa, Artificial Sequence
  Cell adhesion peptide
IDAPS

SEQ ID NO: 14 - protein, 6 aa, Artificial Sequence
  Cell adhesion peptide
KQAGDV

SEQ ID NO: 15 - protein, 5 aa, Artificial Sequence
  Cell adhesion peptide
GRGDS

(19) United States
(12) Patent Application Publication
(10) Pub. No.: US 2024/0052301 A1
(43) Pub. Date: Feb. 15, 2024

(54) MICROCARRIER FOR CELL CULTURE AND CELL CULTURE METHOD

(71) Applicant: SEKISUI CHEMICAL CO., LTD., Osaka (JP)

(72) Inventors: Satoshi HANEDA, Osaka (JP); Hiroki IGUCHI, Osaka (JP)

(73) Assignee: SEKISUI CHEMICAL CO., LTD., Osaka (JP)

(21) Appl. No.: 18/268,476

(22) PCT Filed: Feb. 2, 2022
(86) PCT No.: PCT/JP2022/004061

§ 371 Date: Jun. 20, 2023

(30) Foreign Application Priority Data
Feb. 3, 2021 (JP) 2021-015724

(51) Int. Cl. C12N 5/00

(52) U.S. Cl. CPC C12N 5/0075; C12N 2531/00; C12N 2533/30

(57) ABSTRACT

Provided is a microcarrier for cell culture in which adhesion between microcarriers due to a cell mass can be suppressed. The microcarrier for cell culture according to the present invention includes a base particle and a coating layer covering an outer surface of the base particle, and the coating layer includes a resin having a polyvinyl alcohol derivative skeleton or a poly(meth)acrylic acid ester skeleton and having a peptide moiety, and the microcarrier has an average particle size of 300 μm or more and a CV value of particle size of 10% or less.

### TECHNICAL FIELD

The present invention relates to a microcarrier for cell culture. The present invention also relates to a method for culturing a cell using the microcarrier for cell culture.

### BACKGROUND ART

In research and development in fields of academic study, drug discovery, regenerative medicine, and the like, cells of animals such as humans, mice, rats, pigs, cows, and monkeys have been used. Known methods for culturing a cell include a method in which a microcarrier is used. As a material of a microcarrier, an extracellular matrix (ECM) has been conventionally used.

As shown in Patent Document 1 described below, a microcarrier produced using a synthetic resin also known.

Patent Document 1 below discloses a microcarrier for cell culture including a polymeric microcarrier base formed from copolymerization of a mixture of specific monomers and a polypeptide conjugated to the microcarrier base. In this microcarrier, the equilibrium water content in the microcarrier base is more than 75%.

### RELATED ART DOCUMENT

### Patent Document

Patent Document 1: WO2010/138702A1

### SUMMARY OF THE INVENTION

### Problems to be Solved by the Invention

When conventional microcarriers as described in Patent Document 1 are used for culturing cells, a cell mass is formed between microcarriers and the microcarriers may adhere to each other via the formed cell mass. In this case, the cell mass has a dumpling-like shape, and the efficiency of culturing cells deteriorates.

An object of the present invention is to provide a microcarrier for cell culture in which adhesion between microcarriers due to a cell mass can be suppressed. Furthermore, an object of the present invention is to provide a method for culturing a cell using the microcarrier for cell culture.

### Means for Solving the Problems

According to a broad aspect of the present invention, a microcarrier for cell culture (hereinafter, sometimes described as a microcarrier) is provided that includes a base particle and a coating layer covering an outer surface of the base particle, and the coating layer includes a resin having a polyvinyl alcohol derivative skeleton or a poly(meth)acrylic acid ester skeleton and having a peptide moiety, and the microcarrier has an average particle size of 300 μm or more and a coefficient of variation (CV value) of particle size of 10% or less.

In a specific aspect of the microcarrier according to the present invention, the microcarrier has a water absorption of 10 wt % or less.

In a specific aspect of the microcarrier according to the present invention, the microcarrier has an average particle size of 1000 μm or less.

In a specific aspect of the microcarrier according to the present invention, the polyvinyl alcohol derivative skeleton is a polyvinyl acetal skeleton.

In a specific aspect of the microcarrier according to the present invention, the microcarrier has a specific gravity of 1 g/cm³ or more and 1.2 q/cm³ or less.

In a specific aspect of the microcarrier according to the present invention, the base particle is a resin particle.

In a specific aspect of the microcarrier according to the present invention, the base particle contains a polymer of a monomer having an ethylenic y unsaturated group.

In a specific aspect of the microcarrier according to the present invention, the polymer of a monomer having an ethylenically unsaturated group is an acrylic resin, a divinyibenzene polymer, or a divinyibenzene copolymer.

In a specific aspect of the microcarrier according to the present invention, the peptide moiety has a cell adhesion amino acid sequence.

According to a broad aspect of the present invention, a method for culturing a cell is provided that includes a step of adhering a cell to the above-described microcarrier for cell culture.

### Effect of the Invention

The microcarrier for cell culture according to the present invention includes a base particle and a coating layer covering an outer surface of the base particle, and the coating layer includes a resin having a polyvinyl alcohol derivative skeleton or a poly(meth)acrylic acid ester skeleton and having a peptide moiety. The microcarrier for cell culture according to the present invention has an average particle size of 300 μm or more and a CV value of particle size of 10% or less. The microcarrier for cell culture according to the present invention in ludes the above-described configuration, and therefore in the microcarrier, adhesion between micro carriers due to a cell mass can be suppressed.

### BRIEF DESCRIPTION OF DRAWINGS

FIG. 1 is a sectional view schematically showing a microcarrier for cell culture according to an embodiment of the present invention.

### MODES FOR CARRYING OUT THE INVENTION

Hereinafter, details of the present invention will be described.

### (Microcarrier for Cell Culture)

The microcarrier for cell culture (hereinafter, sometimes abbreviated as “microcarrier”) according to the present invention includes a base particle and a coating layer covering an outer surface of the base particle, and the coating layer includes a resin having a polyvinyl alcohol derivative skeleton or a poly(meth)acrylic acid ester skeleton and having a peptide moiety. The microcarrier according to the present invention has an average particle size of 300 μm or more and a CV value of particle size of 10% or less.

The microcarrier according to the present invention includes the above-described configuration, and therefore in the microcarrier, adhesion between microcarriers due to a cell mass can be suppressed.

As a microcarrier for cell culture, microcarriers have been conventionally used that have a relatively small average particle size (for example, microcarriers having an average particle size of about 100 μm to 200 μm) By using microcarriers having small average particle size, the specific surface area in microcarriers can be increased to increase the area to which cells can adhere. However, the case of conventional microcarriers having a small average particle size, a cell mass is formed between microcarriers, the microcarriers may adhere to each other via the formed cell mass, and as a result, the efficiency of culturing cells deteriorates.

Meanwhile, the microcarrier according to the present invention has a relatively large average particle size and a relatively uniform particle size. The microcarrier according to the present invention includes a base particle and a coating layer including a specific resin. In the micro airier according to the present invention, adhesion between microcarriers due to a cell mass can be suppressed by using the above-described configuration. In addition, in the microcarrier according to the present invention, adhesiveness between the microcarrier and a cell can be enhanced. Furthermore, in the microcarrier according to the present invention, a cell mass having a uniform thickness can be formed on the surface of each microcarrier, and the surface area covered with the cell mass can be increased in each microcarrier. Therefore, in the microcarrier according to the present invention, a high efficiency of culturing cells can be maintained.

The microcarrier according to the present invention does not need to use a natural polymer material such as an extracellular matrix (ECM) as a material, and therefore the microcarrier is inexpensive, less varied among lots, and excellent in safety.

The microcarrier has an average particle size of 300 μm or more. When the microcarrier has an average particle size of less than 300 μm, a cell mass is formed between microcarriers and the microcarriers are likely to adhere to each other via the cell mass.

The microcarrier preferably has an average particle size of 350 μm or more, more preferably 4.00 μm or more, still more preferably 500 μm or more, and particularly preferably 600 μm or more, and preferably 1500 μm or less, more preferably 1000 μm or less, still more preferably 800 μm or less, and particularly preferably 700 μm or less. The microcarrier preferably has an average particle size of 350 μm or more and 1500 μm or less, more preferably 400 μm or more and 1000 μm or less, still more preferably 500 μm or more and 800 μm or less, and particularly preferably 600 μm or more and 700 μm or less. When the average particle size is the above-described lower limit or more, an effect of the present invention can be further effectively exerted. When the average particle size is the above-described upper limit or less, a cell mass having a further uniform thickness can be formed on the surface of each microcarrier. When the average particle size is the above-described upper limit or less, the area to which a cell can adhere can be further increased.

When the microcarrier has a true spherical shape, the particle size of the microcarrier means its diameter, and when the microcarrier has a shape other than a true spherical shape, the particle size of the microcarrier means a diameter of a virtual true sphere having the same volume as the microcarrier.

The average particle size of the microcarrier is preferably a number average particle size. The average particle size of the microcarrier is determined by observing arbitrary microcarriers with an electron microscope or an optical microscope and calculating the average of the particle sizes of the microcarriers, or by using a particle size distribution measuring device. In observation with an electron microscope or an optical microscope, the particle size of one microcarrier is determined in terms an equivalent circle diameter. In observation with an electron microscope or an optical microscope, the average particle size of 50 arbitrary microcarriers as an equivalent circle diameter is substantially equal to the average particle size as a sphere equivalent diameter. With a particle size distribution measuring device, the particle size of one microcarrier is determined as a sphere equivalent diameter. The average particle size of the microcarrier is preferably calculated using a particle size distribution measuring device.

From the viewpoint of exerting an effect of the present invention, the microcarrier has a coefficient of variation (CV value) of particle size of 10% or less.

The microcarrier preferably has a coefficient of variation (CV value) of particle size of 8% or less, more preferably 5% or less, and still more preferably 3% or less. When the coefficient of variation (CV value) is the above-described upper limit or less, an effect of the present invention can be further effectively exerted. The microcarrier may have a coefficient of variation (CV value) of particle size of 0% or more, 0.1% or more, or 1% or more. The microcarrier may have a coefficient of variation (CV value) of particle size of 0% or more and 10% or less, 0.1% or more and 8% or less, 0.1% or more and 5% or less, or 1% or more and 3% or less.

The coefficient of variation (CV value) of particle size of the microcarrier is calculated as follows.

CV value (%)(p/Dn)×100

p: Standard deviation of particle size of microcarrier

Dn: Average particle size of microcarrier

Examples of the method of reducing the coefficient of variation (CV value) of particle size of the microcarrier include a dry classification method and a wet classification method.

The shape of the microcarrier is not particularly limited. The microcarrier may have a spherical shape or a shape other than a spherical shape, and may have a shape such as a flat shape. The spherical shape is not limited to u true spherical shape, and examples of the spherical shape also include substantially spherical shapes, such as a shape having an aspect ratio (long diameter/short diameter) of 1.5 or less.

The microcarrier preferably has a specific gravity of 1 g/cm³ or more and more preferably 1.05 q/cm³ or more, and Preferably 1.2 g/cm³ or less and more preferably 1.15 g/cm³ or less. When the specific gravity is the above-described lower limit or more, the microcarrier suitably settles and its recovery efficiency can be enhanced. When the specific gravity is the above-described upper limit or less, the rotationally by a stirring blade can be improved.

The specific gravity of the microcarrier is measured using a true specific gravity meter.

The microcarrier preferably has a water absorption of 10 wt % or less, more preferably 5 wt % or less, and still more preferably 1 wt % or less. When the water absorption is the above-described upper limit or less, the surface state of the microcarrier is less likely to change at the time of adhesion of cells, resulting in a small variation in the initial fixing rate after cell seeding. When the water absorption is the above-described upper limit or less, cells are less likely to detach from the microcarrier in a medium. The lower limit of the water absorption of the microcarrier is not particularly limited. The water absorption of the microcarrier may be 0 wt % or more, or 0.001 wt % or more.

The water absorption of the microcarrier can be measured as follows.

A microcarrier dried in an oven at 100° C. for 8 hours is prepared. In an environment of a temperature of 37° C. and a relative humidity of 95% RH, 100.0 mg of the microcarrier is left to stand for 24 hours. The weight of the microcarrier after standing is measured. The water absorption of the microcarrier is calculated with the following formula.

Water absorption (wt %)=(W₂−W₁)/W₁×100

W₁: Weight of microcarrier before standing (mg)

W₂: Weight of microcarrier after standing (mg)

Examples of the method of reducing the water absorption of the microcarrier include producing a coating layer using a material having high hydrophobicity.

Hereinafter, the present invention will be specifically described with reference to the drawing.

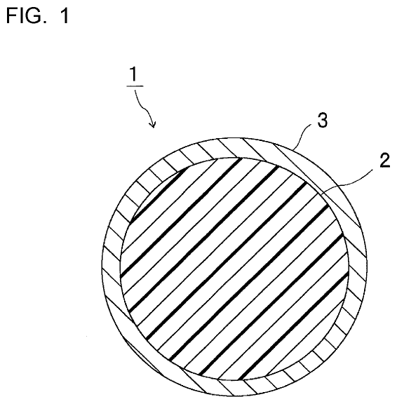

FIG. 1 is a sectional view schematically showing a microcarrier for cell culture according to an embodiment the present invention.

A microcarrier for cell culture 1 shown in FIG. 1 includes a base particle 2 and a coating layer 3 covering an outer surface of the base particle 2. The coating layer 3 is disposed on the surface of the base particle 2 and is in contact with the surface of the base particle 2. The coating layer 3 covers the entire outer surface of the base particle 2, The coating layer 3 includes a resin having a polyvinyl alcohol derivative skeleton or a poly(meth)acrylic acid ester skeleton and having a peptide moiety.

Other details of the microcarrier will be described below.

In the present specification, the term “(meth)acrylate” means one or both of “acrylate” and “methacrylate”, and the term “(meth)acrylic” means one or both of “acrylic” and “methacrylic”.

### (Base Particle)

The material of the base particle is not particularly limited. The material of the base particle is preferably an organic material. The base particle preferably contains a resin. The base particle is preferably a resin particle because a resin particle is easy to manufacture. One kind of the material of the base particle may be used alone, or two or more kinds thereof may be used in combination. One kind of the resin may be used alone, or two or more kinds thereof may be used in combination.

Examples of the organic material (resin) include a polyolefin resin, an acrylic resin, polycarbonate, a polyamide, a phenol formaldehyde resin, a melamine formaldehyde resin, a benzoguanamine formaldehyde resin, a urea formaldehyde resin, a phenol resin, a melamine resin, a benzoguanamine resin, a urea resin, an epoxy resin, an unsaturated polyester resin, a saturated polyester resin, polyethylene terephthalate, polysulfone, polyphenylene oxide, polyacetal, a polyimide, a polyamideimide, polyetheretherketone, polyethersulfone, a divinylbenzene polymer, and a divinyibenzene copolymer.

Examples of the polyolefin resin include polyethylene, polypropylene, polystyrene, polyvinyl chloride, polyvinylidene chloride, polyisobutylene, and polybutadiene.

Examples of the acrylic resin include polymers of monomers such as (meth)acrylic acid, methyl (meth)acrylate, ethyl (meth)acrylate, butyl (meth)acrylate, t-butyl (meth)acrylate, 2-ethylhexyl (meth)acrylate, octyl (meth)acrylate, isopropyl (meth)acrylate, and propyl (meth)acrylate. The acrylic resin may be a homopolymer or a copolymer of the above-described monomers, or a copolymer of the above-described monomers with another monomer. Examples of the acrylic resin include polymethyl methacrylate and polymethyl acrylate.

The material of the base particle is preferably a polymer obtained by polymerizing one or more kinds of polymerizable monomers having an ethylenically unsaturated group. The above-described resin is preferably a polymer of a monomer having an ethylenically unsaturated group. The base particle preferably contains a polymer of a monomer having an ethylenically unsaturated group. In this case, the specific gravity of the base particle can be favorably adjusted, and as a result, the specific gravity of the microcarrier can be adjusted within a suitable range.

Examples of the polymer of a monomer having an ethylenically unsaturated group include an acrylic resin, a divinylbenzene polymer, and a divinylbenzene copolymer. One kind of the monomer having an ethylenically unsaturated group may be used alone, or two or more kinds thereof may be used in combination.

The polymer of a monomer having an ethylenically unsaturated group is preferably an acrylic resin, a divinylbenzene polymer, or a divinylbenzene copolymer. In this case, the specific gravity of the base particle can be favorably adjusted, and as a result, the specific gravity of the microcarrier can be adjusted within a suitable range.

When the base particle contains the polymer of a monomer having an ethylenically unsaturated group, the polymer of a monomer having an ethylenically unsaturated group preferably has a crosslinked structure. In this case, the specific gravity of the base particle can be favorably adjusted, and as a result, the specific gravity of the microcarrier can be adjusted within a suitable range.

Examples of the method for forming the crosslinked structure include the following methods, (1) A method of polymerizing polymerizable components including a monomer having two or more ethylenically unsaturated groups. (2) A method of reacting polymer ((s) of u monomer having an ethylenically unsaturated group with a crosslinking agent to form a crosslinked structure.

In the method (1), examples of the monomer having two or more ethylenically unsaturated groups include divinylbenzene, a polyfunctional (moth) acrylate, triallyl (iso)cyanurate, trimellitate, diallyl phthalate, and diallylacrylamide, One kind of the monomer having two or more ethylenically unsaturated groups may be used alone, or two or more kinds thereof may be used in combination.

In the method (1), the polymerizable components may include another monomer having an ethylenically unsaturated group. Examples of another monomer having an ethylenically unsaturated group include styrene, a monofunctional (meth)acrylate, (met)acrylic acid, acrylonitrile, and vinyl chloride. One kind of another monomer having an ethylenically unsaturated group described above may be used alone, or two or more kinds thereof may be used in combination.

Examples of the polymer obtained with the method (1) include a copolymer of divinylbenzene and styrene, and a copolymer of polyfunctional (meth)acrylate and a monofunctional (meth)acrylate.

Examples of the method (2) include a method of polymerizing a polymerizable components containing a monomer having an ethylenically unsaturated group and a functional group containing active hydrogen in the molecule to obtain polymers and then crosslinking the polymers using a crosslinking agent.

Examples of the functional group including active hydrogen include a hydroxyl group, a carboxyl group, an amino group, and a phenol group. Examples of the monomer including a molecule having an ethylenically unsaturated group and a functional group including active hydrogen include a hydroxyl group-containing (meth)acrylate, (meth)acrylic acid, and an amino group-containing (meth)acrylate. One kind of the monomer including a molecule having an ethylenically unsaturated group and a functional group including active hydrogen may be used alone, or two or more kinds thereof may be used in combination.

The crosslinking agent is not particularly limited as long as it can react with the functional group including active hydrogen, and examples of the crosslinking agent include a polyfunctional isocyanate compound and a polyfunctional epoxy compound, One kind of the crosslinking agent may be used alone, or two or more kinds thereof may be used in combination.

The base particle can be obtained, for example, by polymerizing the monomer having an ethylenically unsaturated group. The polymerization method is not particularly limited, and examples of the polymerization method include known methods such as radical polymerization, ionic polymerization, polycondensation (condensation polymerization), addition condensation, living polymerization, and living radical polymerization. Examples of the polymerization method further include other methods such as suspension polymerization in the presence of a radical polymerization initiator.

The base particle preferably contains a divinylbenzene polymer, a divinyibenzene copolymer, a polystyrene resin, or an acrylic resin, and more preferably contains a divinylbenzene polymer, a divinylbenzene copolymer, or an acrylic resin. The base particle is preferably a divinylbenzene polymer particle, a divinylbenzene copolymer particle, a polystyrene resin particle, or an acrylic resin particle, and more preferably a divinylbenzene polymer particle, a divinylbenzene copolymer particle, or an acrylic resin particle. In this case, the specific gravity of the microcarrier can be suitably controlled.

The content of the resin in 100 wt % of the base particle is preferably 80 wt % or more, more preferably 90 wt % or more, still more preferably 95 wt % or more, even more preferably 97 wt % or more, still even more preferably 99 wt % or more, and most preferably 100 wt % (the whole amount). The content of the resin in 100 wt % of the base particle may be 100 wt % or less or less than 100 wt %.

The base particle preferably has an average particle size of 300 μm or more, more preferably 350 μm or more, still more preferably 400 μm or more, still even more preferably 500 μm or more, and peculiarly preferably 600 μm or more, and preferably 1500 μm or less, more preferably 1000 μm or less, still more preferably 800 μm or less, and particularly preferably 700 μm or less. The base particle preferably has an average particle size of 300 μm or more and 1500 μm or less, more preferably 350 μm or more and 1000 μm or less, still more preferably 400 μm or more and 1000 μm or less, still even more preferably 500 μm or more and 800 μm or less, and particularly preferably 600 μm or more and 700 μm or less. When the average particle size is the above-described lower limit or more, an effect of the present invention can be further effectively exerted. When the average particle size is the above-described upper limit or less, a cell mass having a further uniform thickness can be formed on the surface of each microcarrier.

When the base particle has a true spherical shape, the particle size of the base particle means its diameter, and when the base particle has a shape other than a true spherical shape, the particle size of the base particle means a diameter of a virtual true sphere having the same volume as the base particle.

The average particle size of the base particle is preferably a number average particle size. The average particle size of the base particle is determined by observing arbitrary base particles with an electron microscope or an optical microscope and calculating the average of the particle sizes of the base particles, or by using a particle size distribution measuring device. In observation with an electron microscope or an optical microscope, the particle size of one base particle is determined in terms of an equivalent circle diameter. In observation with an electron microscope or an optical microscope, the average particle size of 50 arbitrary base particles as an equivalent circle diameter is substantially equal to the average particle size as a sphere equivalent diameter. With a particle size distribution measuring device, the particle size of one base particle is determined as a sphere equivalent diameter. The average particle size of the base particle is preferably calculated using a particle size distribution measuring device.

### (Coating Layer)

The microcarrier includes a base particle and a coating layer covering an outer surface of the base particle. The coating layer includes a resin having a polyvinyl alcohol derivative skeleton or a poly(meth)acrylic acid ester skeleton and having a peptide moiety (hereinafter, sometimes described as “resin X”). The resin X has a polyvinyl alcohol derivative skeleton or a poly(meth)acrylic acid ester skeleton and has a peptide moiety. The resin X is a synthetic resin. The coating layer includes the resin X. One kind of the resin X may be used alone, or two or more kinds thereof may be used in combination.

The resin X may have a polyvinyl alcohol derivative skeleton and a peptide moiety, may have a poly(meth)acrylic acid ester skeleton and a peptide moiety, or may have a polyvinyl alcohol derivative skeleton, a poly(meth)acrylic acid ester skeleton, and a peptide moiety.

In the resin X having a polyvinyl alcohol derivative skeleton, the polyvinyl alcohol derivative skeleton is preferably bonded to the peptide moiety via a linker moiety. Therefore, the resin X having a polyvinyl alcohol derivative skeleton preferably has a polyvinyl alcohol derivative skeleton, a peptide moiety, and a linker moiety.

In the resin X having a poly(meth)acrylic acid ester skeleton, the poly (meth)acrylic acid ester skeleton may be bonded to the peptide moiety via a linker moiety, or directly without a linker moiety. The resin X having a poly(meth)acrylic acid ester skeleton may have a poly(meth)acrylic acid ester skeleton, a peptide moiety, and a linker moiety.

### <Polyvinyl Alcohol Derivative Skeleton>

The polyvinyl alcohol derivative skeleton is skeleton portion derived from a polyvinyl alcohol derivative. The polyvinyl alcohol derivative is a compound derived from polyvinyl alcohol. The polyvinyl alcohol derivative is preferably a polyvinyl acetal resin, and the polyvinyl alcohol derivative skeleton is preferably a polyvinyl acetal skeleton, from the viewpoint of further enhancing the adhesiveness between the microcarrier and a cell. That is, the resin X preferably has a polyvinyl acetal skeleton and the peptide moiety. One kind of each of the polyvinyl alcohol derivative and the polyvinyl acetal resin may be used alone, or two or more kinds thereof may be used in combination.

The polyvinyl alcohol derivative skeleton and the Polyvinyl acetal skeleton preferably include a side chain having an acetal group, a hydroxyl group, and an acetyl group. However, the polyvinyl alcohol derivative skeleton and the Polyvinyl acetal skeleton may, for example, have no acetyl group. For example, all acetyl groups in the polyvinyl alcohol derivative skeleton and the polyvinyl acetal skeleton may be bonded to the linker, and thus the polyvinyl alcohol derivative skeleton and the polyvinyl acetal skeleton may have no acetyl group.

The polyvinyl acetal resin can be synthesized through acetalization of polyvinyl alcohol with an aldehyde.

The aldehyde used in acetalization of polyvinyl alcohol is not particularly limited. Examples of the aldehyde include aldehydes having 1 to 10 carbon atoms. The aldehyde may have a chain aliphatic group, a cyclic aliphatic group, or an aromatic group, or may have no such group. The aldehyde may be a chain aldehyde or a cyclic aldehyde. One kind of the aldehyde may be used alone, or two or more kinds thereof may be used in combination.

The aldehyde is preferably formaldehyde, acetaldehyde, propionaldehyde, butyraldehyde, or pentanal, and more preferably butyraldehyde from the viewpoint of further enhancing the adhesiveness between the microcarrier and a cell. Therefore, the polyvinyl acetal resin is more preferably a polyvinyl butyral resin, the polyvinyl acetal skeleton is more preferably a polyvinyl butyral skeleton, and the resin X more preferably has a polyvinyl butyral skeleton.

In the resin X, the polyvinyl alcohol derivative skeleton and the polyvinyl acetal skeleton preferably have an acetalization degree (when the resin X is a polyvinyl butyral resin, a butyralization degree) of 40 mol % or more and more preferably 50 mol % or more, and preferably 90 mol % or less and more preferably 85 mol % or less. When the acetalization degree is the above-described lower limit or more, fixability of a cell can be further enhanced, resulting in efficient cell growth. When the acetalization degree is the above-described upper limit or less, solubility in a solvent can be improved.

In the resin X, the polyvinyl alcohol derivative skeleton and the polyvinyl acetal skeleton preferably have a hydroxyl group content (hydroxyl group amount) of 15 mol % or more and more preferably 20 mol % or more, and preferably mol % or less, more preferably 30 mol % or less, and still more preferably 25 mol % or less.

In the resin X, the polyvinyl alcohol derivative skeleton and the polyvinyl acetal skeleton preferably have an acetylation degree (acetyl group amount) of 1 mol % or more and more preferably 2 mol % or more, and preferably 5 mol % or less and more preferably 4 mol % or less. When the acetylation degree is the above-described lower limit or more and the above-described upper limit or less, the reaction efficiency between the polyvinyl acetal resin and the linker can be enhanced.

The acetalization degree, the acetylation degree, and the hydroxyl group amount of the polyvinyl alcohol derivative skeleton and the polyvinyl acetal skeleton can be measured by ¹H-NMR (nuclear magnetic resonance spectrum).

### <Poly(Meth)Acrylic Acid Ester Skeleton>

The poly(meth)acrylic acid ester skeleton is a skeleton portion derived from a poly(meth)acrylic acid ester. The poly(meth)acrylic acid ester is obtained by polymerizing a (meth)acrylic acid ester. The poly(meth)acrylic acid ester skeleton has a skeleton derived from u (meth)acrylic acid ester, One kind of the poly(meth)acrylic acid ester may be used alone, or two or more kinds thereof may be used in combination.

Examples of the (meth)acrylic acid ester include (meth)acrylic acid alkyl esters, (meth)acrylic acid cyclic alkyl esters, (meth)acrylic acid aryl esters, (meth)acrylic acid polyethylene glycols, and phosphorylcholine (meth)acrylates. One kind of the (meth)acrylic acid ester may be used alone, or two or more kinds thereof may be used in combination.

Examples of the (meth)acrylic acid alkyl esters include methyl (meth)acrylate, ethyl (meth)acrylate, n-propyl (meth)acrylate, isopropyl (meth)acrylate, n-butyl (meth)acrylate, isobutyl (meth)acrylate, t-butyl (meth)acrylate, n-octyl (meth)acrylate, isooctyl (meth)acrylate, 2-ethylhexyl acrylate, (meth)nonyl (meth)acrylate, isononyl (meth)acrylate, decyl (meth)acrylate, isodecyl (meth)acrylate, lauryl (meth)acrylate, stearyl (meth)acrylate, and iso tetradecyl (meth)acrylate.

The (meth)acrylic acid alkyl ester may be substituted with a substituent such as an alkoxy group having 1 to 3 carbon atoms or a tetrahydrofurfuryl group. Examples of such (meth)acrylic acid alkyl ester include methoxyethyl acrylate and tetrahydrofurfLlryl acrylate.

Examples of the (meth)acrylic acid cyclic alkyl esters include cyclohexyl (meth)acrylate and isobornyl (meth)acrylate.

Examples of the (meth)acrylic acid aryl esters include phenyl (meth)acrylate and benzyl (meth)acrylate.

Examples of the (meth)acrylic acid polyethylene glycols include methoxy-polyethylene glycol (meth)acrylate, ethoxy-polyethylene glycol (meth)acrylate, hydroxy-polyethylene glycol (meth)acrylate, methoxy-diethylene glycol (meth)acrylate, ethoxy-diethylene glycol (meth)acrylate, hydroxy-diethylene glycol (meth)acrylate, methoxy-triethylene glycol (meth)acrylate, ethoxy-triethylene glycol (meth)acrylate, and hydroxy-triethylene glycol (meth)acrylate.

Examples of the phosphorylcholine (meth)acrylates include 2-(meth)acryloyloxyeth phosphorylcholine.

The resin X having a poly(meth)acrylic acid ester skeleton preferably has a structural unit derived from a (meth)acrylate compound (A) represented by the formula (A1) or (A2) described below. The poly(meth)acrylic acid ester skeleton preferably has a structural unit derived from a (meth)acrylate compound (A) represented by the formula (A1) or (A2) described below. As a result, the hydrophobicity of the coating layer can be increased, and therefore the water absorption of the microcarrier can be further reduced. Therefore, a variation in the initial fixing rate after cell seeding can be reduced, and cells are less likely to detach from the microcarrier in a medium. The (meth)acrylate compound (A) may contain a (meth)acrylate compound represented by the formula (A1) described below, may contain a (meth)acrylate compound represented by the formula (A2) described below, or may contain both (meth)acrylate compounds represented by the formulae (A1) and (A2) described below. When the (moth) acrylate compound contains both (meth)acrylate compounds represented by the formulae (A1) and (A2), R in the formula (A1) and R in the formula (A2) may be the same or different, One kind of the (meth)acrylate compound (A) may be used alone, or two or more kinds thereof may be used in combination. One kind of each of a (meth)acrylate compound represented by the following formula (A1) and a (meth)acrylate compound represented by the following formula (A2) may be used alone, or two or more kinds thereof may be used in combination.

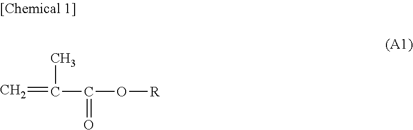

In the formula (A1), R represents a hydrocarbon group having 2 to 18 carbon atoms.

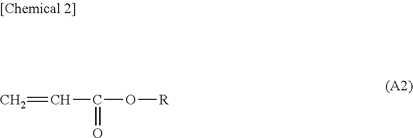

In the formula (A2), R represents a hydrocarbon group having 2 to 18 carbon atoms.

R in the formula (A1) and R in the formula (A2) may each be an aliphatic hydrocarbon group or an aromatic hydrocarbon group, P in the formula. (A1) and R in the formula (A2) are each preferably an aliphatic hydrocarbon group from the viewpoint of improving the solubility of the resin X having a poly(meth)acrylic acid ester skeleton. The aliphatic hydrocarbon group may be linear, may have a branched structure, may have a double bond, or may have no double bond. R in the formula (A1) and R in the formula (A2) may each be an alkyl group or an alkylene group.

The number of carbon atoms in R in the formula (A1) and the number of carbon atoms in R in the formula (A2) are each preferably 4 or more, more preferably 6 or more, still more preferably 8 or more, and particularly preferably 10 or more, and preferably 16 or less, more preferably 14 or less, and most preferably 12. When the numbers of carbon atoms are each the above-described lower limit or more, the hydrophobicity of the resin X can be further increased, and therefore the water absorption of the microcarrier can be further reduced. When the numbers of carbon atoms are each the above-described upper limit or less, the coating property can be enhanced at the time of disposing the material of the coating layer or: the surface of the base particle. In particular when the numbers of carbon atoms are each 12, the water absorption of the microcarrier can be further reduced, and the coating property can be further enhanced.

The (meth)acrylic acid alkyl ester is preferably the (meth)acrylate compound (A).

The resin X having a poly(meth)acrylic acid ester skeleton may have a skeleton derived from a monomer other than a (meth)acrylic acid ester.

Examples of the monomer other than a (meth)acrylic acid ester include (meth)acrylamides and vinyl compounds. One kind of the monomer other than a (meth)acrylic acid ester may be used alone, or two or more kinds thereof may be used in combination.

Examples of the (meth)acrylamides include (meth)acrylamide, N-isopropyl (meth)acrylamide, N-tert-butyl (meth)acrylamide, N,N′-dimethyl (meth)acrylamide, (3-(meth)acrylamidopropyl)trimethyiammonium chloride, 4-(meth)acryloylmorpholine, 3-(meth)acryloyl-2-oxazolidinone, N-[3-(dimethylamino)propyl](meth)acrylamide, N-(2-hydroxyethyl) (meth)acrylamide, N-methylol (meth)acrylamide, and 6-(meth)acrylamidohexanoic acid.

Examples of the vinyl compounds include ethylene, allylamine, vinylpyrrolidone, maleic anhydride, maleimide, itaconic acid, (meth)acrylic acid, and vinylamine.

### <Peptide Moiety>

The peptide moiety is a structural portion derived from a peptide. The peptide moiety has an amino acid sequence. The peptide included in the peptide moiety may be an oligopeptide or a polypeptide, One kind of the peptide may be used alone, or two or more kinds thereof may be used in combination.

The number of amino acid residues in the peptide moiety is preferably 3 or more, more preferably 4 or more, and still more preferably 5 or more, and preferably 10 or less, more preferably 8 or less, and still more preferably 6 or less. When the number of the amino acid residues is the above-described lower limit or more and the above-described upper limit or less, the adhesiveness with a cell after seeding can be further enhanced, and the growth rate of cells can be further enhanced. However, the number of amino acid residues in the peptide moiety may be more than 10, or more than 15.

The peptide moiety preferably has a cell adhesion amino acid sequence. The term “cell adhesion amino acid sequence” refers to an amino acid sequence having a cell adhesion activity confirmed with a phage display method, a Sepharose bead method, or a plate coating method. As the phage display method, for example, the method described in “The Journal of Cell Biology, Volume 130, Number 5, September 1995 1189-1196” can be used. As the Sepharose bead method, for example, the method described in “Protein, nucleic acid and enzyme, Vol, 45, No, 15 (2000) 2477” can be used. As the plate coating method, for example, the method described in “Protein nucleic acid and enzyme, Vol. 45, No, 15 (2000) 2477” can be used.

Examples of the cell adhesion amino acid sequence include an RCM sequence (Arg-Gly-Asp), a YIGSR sequence (Tyi Ile-Gly-Ser-Arg), a PDSGR sequence (Pro-Asp-Ser-Gly-Arg), an HAV sequence (His-Ala-Val), an ADT sequence (Ala-Asp-Thr), a QAV sequence (Gln-Ala-Val), an LDV sequence (Leu-Asp-Val), an IDS sequence (Ile-Asp-Ser), an REDV sequence (Arg-Glu-Asp-Val), an IDAPS sequence (Ile-Asp-Ala-Pro-Ser), a KQAGDV sequence (Lys-Gln-Ala-Gly-Asp-Val), and a TDE sequence (Thr-Asp-Glu). Examples of the cell adhesion amino acid sequence include the sequences described in “Medicina Philosophica, Vol, 9, No. 7, p. 527-535, 1990” and “Osaka Women's and Children's Hospital Journal, Vol. 8, No. 1, p. 58-66, 1992”. The peptide moiety may have only one of the above-described cell adhesion amino acid sequences, or mar have two or more of them.

The cell adhesion amino acid sequence preferably has at least one of the above-described cell adhesion amino acid sequences, more preferably at least the ROD sequence, the YIGSR sequence, or the PDSGR sequence, and still more preferably at least an ROD sequence represented by the formula (1) described below. In this case, the adhesiveness with a cell after seeding can be further enhanced, and the growth rate of cells can be further enhanced.

Arg-Gly-Asp-X Formula (1)

In the above formula (1), X represents Gly, Ala, Val, Ser, Thr, Phe, Met, Pro, or Asn.

The peptide moiety may be linear or may have a cyclic peptide skeleton. The peptide moiety preferably has a cyclic peptide skeleton from the viewpoint of further enhancing the cell growing property. The cyclic peptide skeleton is a cyclic skeleton including a plurality of amino acids. The cyclic peptide skeleton preferably includes 4 or more amino acids and more preferably 5 or more amino acids, and preferably 10 or less amino acids from the viewpoint of further effectively exerting an effect of the present invention.

The resin X preferably has a content of the peptide moiety of 0.1 mol % or more, more preferably 1 mol % or more, still more preferably 5 mol % or more, and particularly preferably 10 mol % or more, and preferably 60 mol % or less, more preferably 50 mol % or less, still more preferably 35 mol % or less, and particularly preferably 25 mol % or less. When the content of the peptide moiety is the above-described lower limit or more, the adhesiveness with a cell after seeding can be further enhanced, and the growth rate of cells can be further enhanced. When the content of the peptide moiety is the above-described upper limit or less, the manufacturing cost can be suppressed. The content (mol %) of the peptide moiety is the substance amount of the peptide moiety with respect to the sum of the substance amounts of the structural units included in the resin X.

The content of the peptide moiety can be measured, for example, by NMR, FT-IR, or LC-MS.

### <Linker Moiety>

The linker moiety is a structural portion derived from a linker. The linker moiety is usually located between the polyvinyl alcohol derivative skeleton or the poly(meth)acrylic acid ester skeleton and the peptide moiety. The polyvinyl alcohol derivative skeleton or the poly(meth)acrylic acid ester skeleton is bonded to the peptide moiety via the linker moiety. The linker moiety is formed using a linker (crosslinking agent). One kind of the linker may be used alone, or two or more kinds thereof may be used in combination.

The linker is preferably a compound having a functional group capable of bonding to the peptide, and more preferably a compound having a functional group capable of condensing with a carboxyl group or an amino group in the peptide.

Examples of the functional group capable of condensing with a carboxyl group or an amino group in the peptide include a carboxyl group, a thiol group, an amino group, a hydroxyl group, and a cyano group.

The linker is preferably a compound having a carboxyl group or an amino group, and more preferably a compound having a carboxyl group from the viewpoint of favorably reacting with the peptide.

When the resin X having a polyvinyl alcohol derivative skeleton is obtained, examples of the linker having a carboxyl group include (meth)acrylic acid and carboxyl group-containing acrylamide. When a carboxylic acid (carboxylic acid monomer) having a polymerizable unsaturated group is used as the linker having a carboxyl group, the carboxylic acid monomer can be polymerized by graft polymerization at the time of introducing the linker, resulting in an increase the number of carboxyl groups that can react with the peptide.

The linker is preferably (meth)acrylic acid, and more preferably acrylic acid from the viewpoint of bonding the polyvinyl alcohol derivative and the peptide favorably.

When the resin X having a poly(meth)acrylic acid ester skeleton is obtained, the linker preferably has a functional group capable of bonding to a (meth)acrylic acid ester. Examples of the functional group capable of bonding to a (meth)acrylic acid ester include a vinyl group, a (meth)acryloyl group, and an allyl group. The linker more preferably has a (meth)acryloyl group as the functional group capable of bonding to a (meth)acrylic acid ester, and is preferably a compound having a carboxyl group or an amino group and having a (meth)acryloyl group.

When the resin. X having a poly (meth)acrylic acid ester skeleton is obtained, examples of the linker include (meth)acrylic acid, itaconic acid, and acrylamide.

The linker is preferably (meth)acrylic acid or itaconic acid, and more preferably (meth)acrylic acid from the viewpoint of bonding the poly(meth)acryl acid ester and the peptide favorably.

### <Other Details of Coating Layer>

The resin X preferably has a weight average molecular weight of 10,000 or more and more preferably 50,000 or more, and preferably 1,200,000 or less and more preferably 600,000 or less. When the weight average molecular weight is the above-described lower limit or more and the above-described upper limit or less, an effect of the present invention can be further effectively exerted. When the weight average molecular weight is the above-described upper limit or less, the extension of cells in cell culture can be further effectively enhanced.

The resin X having a polyvinyl alcohol derivative skeleton preferably has a weight average molecular weight of 10,000 or more and more preferably 50,000 or more, and preferably 1,200,000 or less and more preferably 600,000 or less, when the weight average molecular weight is the above-described lower limit or more and the above-described upper limit or less, an effect of the present invention can ne further effectively exerted. When the weight average molecular weight is the above-described upper limit or less, the extension of cells in cell culture can be further effectively enhanced.

The resin X having a poly(meth)acrylic acid ester skeleton preferably has a weigh; average molecular weight of or more and more preferably 50,000 or more, and preferably 1,200,000 or less and more preferably 600,000 or less. When the weight average molecular weight is the above-described lower limit or more and the above-described upper limit or less, an effect of the present invention can ne further effectively exerted. When the weight average molecular weight is the above-described upper limit or less, the extension of cells in cell culture can be further effectively enhanced.

The weight average molecular weight of the resin. X: can be measured, for example, with the following method. The resin X is dissolved in tetrahydrofuran (THF) to prepare a 0.2 wt % solution of the resin X, Next, evaluation is performed under the following measurement conditions using a gel permeation chromatography (GPC) measuring device (APC System, manufactured by Waters Corporation).
Column: HSPgel HR MB-M 6.0×150 mm
Flow rate: 0.5 mL/min
Column temperature: 40° C.
Injection volume: 10 μL
Detector: RI, PDA
Standard sample: polystyrene

The coating layer may include only the resin X, The coating layer may include a component other than the resin X. Examples of the component other than the resin X include resins other than the resin X. Examples of the component other than the resin X include polyvinyl alcohol derivatives such as a polyvinyl acetal resin, poly(meth)acrylic acid esters, polyolefin resins, polyether resins, a polyvinyl alcohol resin, polyesters, epoxy resins, polyamide resins, polyimide resins, polyurethane resins, a polycarbonate resin, cellulose, and polypeptides, One kind of the component other than the resin X may be used alone, or two or more kinds thereof may be used in combination.

The coating layer may have on a layer including the resin X. The coating layer may have a layer including no resin X and a layer including the resin X. When the coating layer has a layer including no resin X and a layer including the resin X, the coating layer preferably has the layers so that the layer including no resin is located on the base particle side and the layer including the resin X is located outside the layer including no resin X In this case, the adhesiveness between the microcarrier and a cell can be further enhanced.

The resin X is preferably present at least on the outer surface of the microcarrier. The outermost layer of the microcarrier is preferably a layer including the resin X. In this case, the adhesiveness between the microcarrier and a cell can be further enhanced.

The content of the resin X in 100 wt % of the layer including the resin X is preferably 90 wt % or more, more preferably 95 wt % or more, still more preferably 97.5 wt % or more, particularly preferably 99 wt % or more, and most preferably 100 wt % (the whole amount), When the content of the resin X is the above-described lower limit or more, an effect of the present invention can be further effectively exerted.

The surface area covered with the coating layer in 100% of the total surface area of the base particle (coverage) is preferably 50% or more, more preferably 70% or more, still more preferably 90% or more, still even more preferably 95% or more, particularly preferably 99% or more, and most preferably 100%. When the coverage is the above-described lower limit or more, the adhesiveness between the microcarrier and a cell can be further enhanced, and an effect of the present invention can be further effectively exerted. The coverage may be 100% or less, less than 100%, or 99% or less.

The coverage is determined by observing the microcarrier with an electron microscope or an optical microscope and calculating the percentage of the surface area covered with the coating layer with respect to the projected area of the base particle.

The thickness of the coating layer is preferably 10 nm or more and more preferably 50 nm or more, and preferably μm or less and more preferably 500 nm or less. When the thickness of the coating layer is the above-described lower limit or more and the above-described upper limit or less, the adhesiveness between the microcarrier and a cell can be further enhanced. When the thickness of the coating layer is the above-described lower limit or more and the above-described upper limit or less, an effect of the present invention can be further effectively exerted.

The thickness of the coating layer can be measured, for example, by observing a cross section of the microcarrier using a scanning electron microscope (SEM), For calculation of the thickness of the coating layer, the average of the thicknesses of five arbitrary parts of the coating layer is preferably calculated as the thickness of the coating layer of one microcarrier, and the average of the thickness of the entire coating layer is more preferably calculated as the thickness of the coat, layer of one microcarrier. The thickness of the coating layer is preferably determined by calculating the average of the thicknesses of the coating layers of 50 arbitrary microcarriers.

Examples of the method of obtaining the resin X having a polyvinyl alcohol derivative skeleton include the following methods.

A polyvinyl alcohol derivative (for example, a Polyvinyl acetal resin) is reacted with a linker to obtain a reaction product in which the polyvinyl acetal resin and the linker are bonded. The obtained reaction product is reacted with a peptide to obtain a resin X: having a polyvinyl alcohol derivative skeleton (polyvinyl acetal skeleton).

Examples of the method of obtaining the resin X having a poly(meth)acrylic acid ester skeleton include the following methods.

A monomer including a (meth)acrylic acid ester is polymerized to obtain an acrylic resin. The obtained acrylic resin is reacted with a peptide and a inker that is used as necessary to obtain a resin X having a poly(meth)acrylic acid ester skeleton.

Examples of the method of obtaining a resin X having the polyvinyl alcohol derivative skeleton and the poly(meth)acrylic acid ester skeleton include the following methods.

A resin having the polyvinyl alcohol derivative skeleton and the poly(meth)acrylic acid ester skeleton is obtained with the following method (i), (ii), or (iii). (i) A polyvinyl acetal resin is synthesized using polyvinyl alcohol in which an acrylic acid ester is copolymerized. (ii) A polyvinyl acetal resin is synthesized using polyvinyl alcohol and polyvinyl alcohol in which an acrylic acid ester is copolymerized, (iii) An acrylic acid ester is graft-copolymerized with a polyvinyl acetal resin. The resin obtained with the method (i), (ii), or (iii), a peptide, and a linker that is used as necessary are reacted to obtain a resin X having the polyvinyl alcohol derivative skeleton and the poly(meth)acrylic acid ester skeleton.

Examples of the method of obtaining the microcarrier by disposing the coating layer on the surface of the base particle include the following methods (1) and (2).

Method (1): The resin X obtained with the above-described method is dissolved in a solvent to obtain a resin X-containing solution. The resin X-containing solution is sprayed onto the base particle, or the base particle impregnated with the resin X-containing solution is separated, and thus a microcarrier can be produced that includes a layer (coating layer) including the resin X on the outer surface of the base particle.

Method (2): A resin having no polyvinyl alcohol derivative skeleton or no poly(meth)acrylic acid ester skeleton (resin X before bonding with a peptide) is prepared. This resin is dissolved in a solvent to obtain a resin-containing solution. The resin-containing solution is sprayed onto the base particle, or the base particle impregnated with the resin-containing solution is separated, and thus a particle is obtained in which a layer including no resin X (a layer including a polyvinyl alcohol derivative or a poly(meth)acrylic acid ester) is disposed on the outer surface of the base particle. The obtained particle is subjected to the above-described method to reacted the polyvinyl alcohol derivative or the poly(meth)acrylic acid ester in the layer including no resin X, a peptide, and a linker that is used as necessary. Thus, a microcarrier can be produced that includes a layer including no resin X and a layer including the resin X as coating layers on the outer surface of the base particle.

### (Other Details of Microcarrier)

The microcarrier is used for culturing a cell.

Examples of the cell include cells of animals such as humans, mice, rats, pigs, cows, and monkeys. Examples of the cell include somatic cells, and include stem cells, progenitor cells, and mature cells. The somatic cells may be cancer cells.

Examples of the stem cells include mesenchymal stem cells (MSCs), induced pluripotent stem (iPS) cells, embryonic stem (ES) cells, multilineage-differentiating stress-enduring (Muse) cells, embryonic carcinoma cells, embryonic germ stem cells, and multipotent germline stem (mGS) cells.

Examples of the mature cells include nerve cells, cardiomyocytes, retinal cells, and hepatocytes.

The microcarrier is preferably used for three-dimensional cell culture. In two-dimensional culture, cells are cultured on a plane such as a plate. Meanwhile, three-dimensional culture is a culture method in which cells are cultured so that they have a thickness also in the longitudinal direction.

The microcarrier is preferably used for culture with a serum-free medium. Because the microcarrier includes the resin X, the adhesiveness with a cell can be enhanced even in culture with a serum-free medium containing no feeder cell or no adhesive protein, and in particular, the initial fixing rate after cell seeding can be further enhanced. Furthermore, because the microcarrier includes the resin X, an effect of the present invention can be exerted even in culture with a serum-free medium.

The microcarrier preferably includes substantially no material derived from an animal. When no material derived from an animal is included, a microcarrier can be provided that has high safety and little variation in Quality at the time of manufacturing. The phrase “substantially no material derived from an animal is included” means that the microcarrier has a content of materials derived from an animal of 3 wt % or less. The microcarrier preferably has a content of materials derived from an animal of 1 wt % or less, and most preferably 0 wt %. That is, the microcarrier most preferably includes no material derived from an animal.

### (Method for Culturing Cell)

A cell can be cultured using the microcarrier. The method for culturing a cell according to the present invention is a method for culturing a cell using the above-described microcarrier. Examples of the cell include the above-described cells.

The method for culturing a cell preferably includes a step of adhering a cell to the microcarrier. The cell may be a cell mass. The cell mass can be obtained by adding a cell dissociation agent to a culture vessel in which culture is confluent and crushing the mixture uniformly by pipetting. The cell dissociation agent is not particularly limited, but an ethylenediamine/phosphate buffer solution is preferable. The cell mass preferably has a size of 50 μm to 200 μm.

Hereinafter, the present invention will be described in more detail with reference to Examples and Comparative Examples. The present invention is not limited to these Examples.

The content of a structural unit in the obtained resin was measured by 1H-NMR (nuclear magnetic resonance spectrum) after dissolving the synthetic resin in dimethyl sulfoxide (DMSO-d6).

### Example 1

### (1) Production of Base Particle A

A mixed liquid was obtained by mixing 800 parts by weight of divinylbenzene (purity: 57%) and 200 parts by weight of styrene. To the obtained mixed liquid, 20 parts by weight of benzoyl peroxide was added, and the resulting mixture was stirred until the benzoyl peroxide was uniformly dissolved to obtain a monomer mixed solution. Into a reaction tank, 4000 parts by weight of a 2 wt % aqueous solution was put that was obtained by dissolving polyvinyl alcohol having a molecular weight of about 1700 in pure water. Next, the obtained monomer mixed solution was put into the reaction tank, and the resulting mixture was stirred for 4 hours to adjust the particle size of a droplet of the monomer to a predetermined particle size. Next, a reaction was performed in a nitrogen atmosphere at 85° C. for 9 hours to perform a polymerization reaction in droplets of the monomer, and thus particles were obtained. The obtained particles were washed with each of hot water, methanol, and acetone several times, and then classified to obtain a base particle A having an average particle size of 600 μm and a CV value of particle size of 1%. The base particle A is a resin particle of a divinylbenzene copolymer (described as DVB in the tables).

(2) Production of polyvinyl acetal resin

Into a reactor equipped with a stirrer, 2700 mL of ion-exchanged water, 300 parts by weight of polyvinyl alcohol having an average polymerization degree of 1700 and a saponification degree of 99 mol % were put, and heated and dissolved while stirred to obtain a solution. To the obtained solution, 35 wt % hydrochloric acid was added as a catalyst so that the hydrochloric acid concentration was 0.2 wt %. Next, the temperature was adjusted to 15° C., and 22 parts by weight of n-butyraldehyde was added while the solution was stirred, Next, 148 parts by weight of n-butyraldehyde was added to precipitate a white particulate polyvinyl acetal resin (polyvinyl butyral resin), After a apse of 15 minutes from the precipitation, 35 wt % hydrochloric acid was added so that the hydrochloric acid concentration was 1.8 wt %, then the solution was heated to and held at 50° C. for 2 hours. Next, the solution was cooled and neutralized, and then the polyvinyl butyral resin was washed with water and dried to obtain a polyvinyl acetal resin polyvinyl butyral resin, average polymerization degree: 1700, acetalization degree (butyralization degree) mol %, hydroxyl group amount: 27 mol %, acetylation degree: 3 mol %).

### (3) Formation of Linker Moiety

In 300 parts by weight of tetrahydrofuran (THF), 99 parts by weight of the obtained polyvinyl acetal resin and 1 part by weight of acrylic acid (linker) were dissolved, the resulting solution was reacted under ultraviolet irradiation for 20 minutes in the presence of a photoradical Polymerization initiator, and thus the polyvinyl acetal resin and the acrylic acid were graft-copolymerized to form a linker moiety.

(4) Production of Particle Covered with polyvinyl acetal resin in which Linker Moiety Formed

In 19 parts by weight of butanol, 1 part by weight of the polyvinyl acetal resin in which the linker moiety was formed was dissolved. To the obtained solution, 1 part by weight of the base particle A was added, the resulting mixture was stirred and then filtered, and the filtered Particle was washed with pure water and vacuum-dried at 60° C. for 5 hours to obtain a particle covered with the polyvinyl acetal resin in which the linker moiety was formed.

### (5) Production of Microcarrier

A linear peptide having an amino acid sequence of Gly-Arg-Gly-Asp-Ser (the number of amino acid residues: 5) was Prepared. To phosphate buffered saline containing neither calcium nor magnesium, 1 part by weight of this peptide and 1 part by weight of 1-ethyl-3-(3-dimethylaminopropy)carbodiimide hydrochloride (condensing agent) were added so that the final peptide concentration was 1 mM, and thus a peptide-containing liquid was produced. To 20 parts by weight of the obtained peptide-containing liquid, 1 part by weight of the particle covered with the polyvinyl acetal resin in which the linker moiety was formed was added, and thus the carboxyl group in the linker moiety and the amino group of Gly of the peptide were dehydrated and condensed. The obtained suspension was filtered, washed with pure water, and vacuum-dried at 60° C. for 5 hours to obtain a microcarrier. In the tables, the resin X having a polyvinyl alcohol derivative skeleton (polyvinyl acetal skeleton) obtained with the above-described method is described as resin X1. The resin X1 has an amino acid sequence of Cllr-Arg-Gly-Asp-Ser as a peptide moiety.

### Example 2

### (1) Production of Base Particle B

A polymerization reaction was performed in the same manner as in Example 1 to obtain particles. The obtained particles were classified to obtain a base particle 3 having an average particle size of 350 μm and a CV value of particle size of 1%.

### (2) Production of Microcarrier

A microcarrier was produced in the same manner as in Example 1 except that the obtained base particle B was used.

### Example 3

### (1) Production of Base Particle C

A polymerization reaction was performed in the same manner as in Example 1 to obtain particles. The obtained particles were classified to obtain a base particle C having an average particle size of 900 μm and a CV value of particle size of 1%.

### (2) Production of Microcarrier

A microcarrier was produced in the same manner as in Example 1 except that the obtained base particle C was used.

### Example 4

### (1) Production of Base Particle D

A polymerization reaction was performed in the same manner as Example 1 to obtain particles. The obtained particles were classified to obtain a base particle D having an average particle size of 600 μm and a CV value of particle size of 8%.

### (2) Production of Microcarrier

A microcarrier was produced in the same manner as in Example 1 except that the obtained base particle D was used.

### Example 5

### (1) Base Particle

As the base particle, the base particle A was used.

### (2) Production of Acrylic Resin

In 30 parts by weight of tetrahydrofuran, 29 parts by weight of butyl acrylate, 3 parts by weight of 2-hydroxyethyl acrylate, and 1 part by weight of acrylic acid were dissolved to obtain an acrylic monomer solution. In the obtained acrylic monomer solution, 1 part by weight of Irgacure 184 (manufactured by BASF SE) was dissolved, and the obtained liquid was applied onto a PET film. The applied product was irradiated with light having a wavelength of 365 nm at an integrated light amount of 2000 mJ/cm² using a IN conveyor (“ECS 301G1” manufactured by EYE GRAPHICS CO., LTD.) at 25° C. to obtain an acrylic resin solution. The obtained acrylic resin solution was vacuum-dried at 80° C. for 3 hours to obtain an acrylic resin. The obtained acrylic resin had a linker moiety. The obtained acrylic resin had a weight average molecular weight of about 100,000.

(3) Production of Particle Covered with acrylic resin

In 19 parts by weight of butanol, 1 part by weight of the obtained acrylic resin was dissolved. To this solution, 1 part by weight of the base particle A was added, the resulting mixture was stirred and then filtered, and the filtered particle was washed with pure water and vacuum-dried at 60° C. for 5 hours to obtain a particle covered with the acrylic resin.

### (4) Production of Microcarrier

A microcarrier was obtained in the same manner as in Example 1 except that the obtained particle covered with the acrylic resin was used. In the table, the resin X having a poly(meth)acryl c acid ester skeleton obtained with the above-described method is described as resin X2. The resin X2 has an amino acid sequence of Gly-Arg-Gly-Asp-Ser as a peptide moiety.

### Example 6

### (1) Production of Base Particle E

A polymerization reaction was performed in the same manner as in Example 1 to obtain particles. The obtained particles were classified to obtain a base particle E having an average particle size of 1500 μm and a CV′ value of particle size of 1%.

### (2) Production of Microcarrier

A microcarrier was produced in the same manner as in Example 1 except that the obtained base particle E was used.

### Example 7

As the base particle, the base particle A was used.

(2) Production of acrylic resin

In 27 parts by weight of tetrahydrofuran, 10 parts by weight of dodecyl acrylate and 2.7 part by weight of acrylic acid were dissolved to obtain an acrylic monomer solution. In the obtained acrylic monomer solution, 0.0575 parts by weight of Irgacure 184 (manufactured by EASF SE) was dissolved, and the obtained liquid was applied onto a PET film. The applied product was irradiated with light having a wavelength of 365 nm at an integrated light amount of 2000 mJ/cm² using a UV conveyor (“ECS 301G1” manufactured by EYE GRAPHICS CO., LTD.) at 25° C. to obtain a (meth)acrylic copolymer solution. The obtained (meth acrylic copolymer solution was vacuum-dried at 80° C. for 3 hours to obtain an acrylic resin having a linker moiety.

(3) Production of Particle Covered with Acrylic Resin

In 19 parts by weight of butanol, 1 part by weight of the obtained acrylic resin was dissolved. To this solution, 1 part by weight of the base particle was added, the resulting mixture was stirred and then filtered, and the filtered particle was washed with pure water and vacuum-dried at 60° C. for 5 hours to obtain a particle covered with the acrylic resin.

### (4) Production of Microcarrier

The obtained particle covered with the acrylic resin was used. In addition, a cyclic peptide having an amino acid sequence of Arg-Gly-Asp-Phe-Lys (the number of amino acid residues was 5, Arg and Lys were bonded to form a cyclic skeleton, and Phe was a D-form) was prepared as a peptide. A microcarrier was obtained in the same manner as in Example 1 except that this peptide was used to dehydrate and condense the carboxyl group in the structural unit derived from acrylic acid of the acrylic resin and the amino group of Lys of the peptide. In the table, the resin X having a poly(meth)acrylic acid ester skeleton obtained with the above-described method is described as resin X3. The resin X3 has an amino acid sequence of Arg-Gly-Asp-Phe-Lys (cyclic peptide skeleton) as a peptide moiety.

### Example 8

### (1) Production of Base Particle F

Micropearl GS-L300 (manufactured by SEKISUI CHEMICAL CO., LTD., average particle size: 300 μm, CV value of particle size: 7%, polyfunctional acrylic resin particles) was prepared. The particles were classified to obtain a base particle F having an average particle size of 300 μm and a CV value of particle size of 10. The base particle F is a resin particle of an acrylic resin (described as ACE in the table).

### (2) Production of Microcarrier

A microcarrier was produced in the same manner as in Example 1 except that the obtained base particle F was used.

### Comparative Example 1

(1) Production of base particle G

A polymerization reaction was performed in the same manner as in Example 1 to obtain particles. The obtained particles were classified to obtain a base particle C having an average particle size of 600 μm and a CV value of particle size of 15%—.

### (2) Production of Microcarrier

A microcarrier was produced in the same manner as Example 1 except that the obtained base particle C was used.

### Comparative Example 2

(1) Production of base particle H

A polymerization reaction was performed in the same manner as in Example 1 to obtain particles. The obtained particles were classified to obtain a base particle H having an average particle size of 100 μm and a CV value of particle size of 1%.

### (2) Production of Microcarrier

A microcarrier was produced in the same manner as in Example 1 except that the obtained base particle H was used.

### (Evaluation)

### (1) Average Particle Size and Coefficient of Variation (CV Value) of Particle Size of Microcarrier

The obtained microcarriers were observed with a scanning electron microscope. The average particle size in terms of equivalent circle diameter and the coefficient of variation (CV value) of particle size of 50 arbitrary microcarriers were calculated.

### (2) Thickness of Coating Layer

Sections of the obtained microcarriers were observed with a scanning electron microscope. For each of 50 arbitrary microcarriers, the thickness of the coating layer was measured, and the average of the thicknesses was taken as the thickness of the coating layer of the microcarriers.

### (3) Specific Gravity of Microcarrier

The specific gravity of the obtained microcarrier was measured in a dry state under an argon gas atmosphere using a true specific gravity meter (“AccuPyc II” manufactured by SHIMADZU CORPORATION).

### (4) Water Absorption of Microcarrier

The obtained microcarrier was dried in an oven at 100° C. for 8 hours. This microcarrier (100.0 mg) was weighed, and let, to stand for 24 hours in an environment of a temperature of 37° C. and a relative humidity of 95% RH. The weight of the microcarrier after standing was measured, and the water absorption of the microcarrier was calculated with the following formula.

Water absorption (wt %)=(W₂−W₁)/W₁×100

W₁: Weight of microcarrier before standing (mg)

W₂: Weight of microcarrier after standing (mg)

### (5) Evaluation of Cell Culture

In one well of a 12-well plate (manufactured by Corning incorporated, with an untreated flat bottom), 80 mg of the obtained microcarrier was weighed to obtain a culture plate.

The following liquid medium and ROCK (Rho-associated kinase)-specific inhibitor were prepared.

TeSR E8 medium (manufactured by STEMCELL Technologies)

ROCK-Inhibitor (Y27632)

The h-iPS cells 253(31 in a confluent state and 1 mL of a 0.5 mM ethylenediaminetetraacetic acid/phosphate buffer solution were added into a. (05 mm dish, and the dish was allowed to stand at room temperature for 5 minutes. The ethylenediaminetetraacetic acid/phosphate buffer solution was removed, and then pipetting was performed with 1 mL of the liquid medium to obtain a cell suspension. The obtained cell suspension including 1.0×10 4 cells was seeded in the culture plate containing 1 mL of the liquid medium.

The microcarriers subjected to culture for 5 days were photographed with a phase contrast microscope.

In 20 arbitrary microcarriers in which cell adhesion was observed, adhesion between microcarriers due to a cell mass was evaluated in accordance with the following criteria.

### <Evaluation Criteria for Adhesion Between Microcarriers>

AA: Twenty microcarriers include less than 5 microcarriers in which adhesion between microcarriers due to a cell mass is observed.

A: Twenty microcarriers include 5 or more and less than 8 microcarriers in which adhesion between microcarriers due to a cell mass is observed.

B: Twenty microcarriers include 8 or more and less than 10 microcarriers in which adhesion between microcarriers due to a cell mass is observed.

C: Twenty microcarriers include 10 or more microcarriers in which adhesion between microcarriers due to a cell mass is observed.

In 20 arbitrary microcarriers in which cell adhesion was observed, the uniform coverage by cells adhered to the microcarriers was evaluated in accordance with the following criteria.

### <Evaluation Criteria for Uniform Coverage by Cells>

AA: Twenty microcarriers include 10 or more microcarriers in which 90% or more of the surface of each microcarrier is covered with a cell mass.

A: The criterion “AA” is not satisfied, and 20 microcarriers include 10 or more microcarriers in which 70% or more and less than 90% of the surface of each microcarrier is covered with a cell mass.

B: The criteria “AA” and “A” are not satisfied, and 20 microcarriers include 10 or more microcarriers in which 50% or more and less than 70% of the surface of each microcarrier is covered with a cell mass.

The criteria “AA”, “A”, and “B” are not satisfied, and 2C microcarriers include 10 or more microcarriers in which less than 50% of the surface of each microcarrier is covered with a cell mass.

Tables 1 and 2 described below show the details and the results.

TABLE 1

| | | | Example 1 | Example 2 | Example 3 | Example 4 | Example 5 |
|---|---|---|---|---|---|---|---|
| Base particle | Kind | | A | B | C | D | A |
| | Kind | | DVB | DVB | DVB | DVB | DVB |
| | Average particle size | μm | 600 | 350 | 900 | 600 | 600 |
| | Specific gravity | g/cm³ | 1.11 | 1.11 | 1.11 | 1.11 | 1.11 |
| Coating layer | Kind of resin X | | Resin X1 | Resin X1 | Resin X1 | Resin X1 | Resin X2 |
| | Thickness | nm | 100 | 100 | 100 | 100 | 100 |
| Microcarrier | Average particle size | μm | 600 | 350 | 900 | 600 | 600 |
| | CV value of particle size | % | 1 | 1 | 1 | 8 | 1 |
| | Specific gravity | g/cm³ | 1.11 | 1.11 | 1.11 | 1.11 | 1.11 |
| | Water absorption | wt % | 0.5 | 0.5 | 0.5 | 0.5 | 0.5 |

| | | | | | | |
|---|---|---|---|---|---|---|
| Evaluation of | Adhesion between microcarriers | AA | A | AA | A | AA |
| cell culture | Uniform coverage by cells | AA | AA | A | AA | AA |

TABLE 2

| | | | | | | Comparative | Comparative |
|---|---|---|---|---|---|---|---|
| | | | Example 6 | Example 7 | Example 8 | Example 1 | Example 2 |
| Base particle | Kind | | E | A | F | G | H |
| | Kind | | DVB | DVB | ACR | DVB | DVB |
| | Average particle size | μm | 1500 | 600 | 300 | 600 | 100 |
| | Specific gravity | g/cm³ | 1.11 | 1.11 | 1.19 | 1.11 | 1.11 |
| Coating layer | Kind of resin X | | Resin X1 | Resin X3 | Resin X1 | Resin X1 | Resin X1 |
| | Thickness | nm | 100 | 100 | 100 | 100 | 100 |
| Microcarrier | Average particle size | μm | 1500 | 600 | 300 | 600 | 100 |
| | CV value of particle size | % | 1 | 1 | 1 | 15 | 1 |
| | Specific gravity | g/cm³ | 1.11 | 1.11 | 1.19 | 1.11 | 1.11 |
| | Water absorption | wt % | 0.5 | 0.5 | 0.5 | 0.5 | 0.5 |

| | | | | | | |
|---|---|---|---|---|---|---|
| Evaluation of | Adhesion between microcarriers | AA | AA | A | C | C |
| cell culture | Uniform coverage by cells | B | AA | A | AA | AA |

### EXPLANATION OF SYMBOLS

1: Microcarrier for cell culture
2: Base particle
3: Coating layer

1. A microcarrier for cell culture, the microcarrier comprising:
a base particle; and
a coating layer covering an outer surface of the base particle,
the coating layer including a resin having a polyvinyl acetal skeleton, the resin having a peptide moiety,
the microcarrier having an average particle size of 300 μm or more, and
having a coefficient of variation (CV value) of particle size of 10% or less.

2. The microcarrier for cell culture according to claim 1, the microcarrier having a water absorption of 10 wt % or less.

3. The microcarrier for cell culture according to claim 1, the microcarrier having an average particle size of 1000 μm or less.

4. The microcarrier for cell culture according to claim 1, wherein the polyvinyl acetal skeleton is a polyvinyl butyral skeleton.

5. The microcarrier for cell culture according to claim 1, the microcarrier having a specific gravity of 1 g/cm³ or more and 1.2 g/cm³ or less.

6. The microcarrier for cell culture according to claim 1, wherein the base particle is a resin particle.

7. The microcarrier for cell culture according to claim 1, wherein the base particle contains a polymer of a monomer having an ethylenically unsaturated group.

8. The microcarrier for cell culture according to claim 7, wherein the polymer of a monomer having an ethylenically unsaturated group is an acrylic resin, a divinylbenzene polymer, or a divinylbenzene copolymer.

9. The microcarrier for cell culture according to claim 1, wherein the peptide moiety has a cell adhesion amino acid sequence.

10. A method for culturing a cell, the method comprising a step of adhering a cell to the microcarrier for cell culture according to claim 1.
